(19) United States
(12) Patent Application Publication
(10) Pub. No.: US 2012/0052050 A1
(43) Pub. Date: Mar. 1, 2012

(54) USE OF SPECIFIC LACTIC BACTERIA FOR THE PREPARATION OF IMMUNOMODULATING COMPOSITIONS

(75) Inventors: Giancarla DONDI, Novara (IT); Patrizia Malfa, Novara (IT)

(73) Assignee: PROGE FARM S.R.L., Novara (IT)

(21) Appl. No.: 13/292,506

(22) Filed: Nov. 9, 2011

Related U.S. Application Data
(62) Division of application No. 11/989,761, filed on Mar. 18, 2008.

(30) Foreign Application Priority Data
Aug. 2, 2005 (IT) MI2005A001510

(51) Int. Cl. A61K 35/74; A61P 31/04; A61P 37/06

(52) U.S. Cl. 424/93.45

(57) ABSTRACT

The invention concerns the use of two lactobacteria for the preparation of immunomodulating compositions, in particular for the treatment and/or prevention of allergies and immunodeficiencies, and specifically use of the following two strains Lactobacillus salivarlus I 1794 and Lactobacillus paracasei I 1688, alone or in combination, in the treatments indicated above.

### FIELD OF THE INVENTION

The present invention concerns the use of some particular lactobacteria for the preparation of immunomodulating compositions, more specifically the invention concerns use of the following two strains Lactobacillus salivarius I 1794 and Lactobacillus paracasei I 1688, alone or mixed with each other.

### TECHNICAL BACKGROUND

The influence of certain bacteria, that produce lactic acid (LAB), present mainly in fermented food and probiotic preparations, on the immunity of animals and humans has been described. In particular, some LABs have proved to be capable of interacting with the immune system, modifying the type and degree of protection against pathogens and tumoural degenerations; an increase in the immune response at the level of the mucous membranes has also been demonstrated (Int. I.

Immunopathol. Pharmacol., 2004; 17:127-134; infection and Immunity, 2000; 68(2):752-59).

### DESCRIPTION OF THE INVENTION

It is also known that not all lactobacteria are probiotic and that only some have an effect on the immune system. In vitro immunology studies conducted with various strains of LAB have provided conflicting and in some respects antithetic results, demonstrating that not only is the existence of an activity of the LABs on the immune system non-predictable but also that some strains are able to inhibit the activity of other species of the same genus (J. Immunol., 2002, 168:171-178).

It has now been found that two specific strains of lactobacteria have a powerful immunomodulator effect, alone or combined, and are therefore useful for the treatment of pathologies associated with alterations of the immune system.

In particular it has surprisingly been found that two strains of lactobacteria Lactobacillus salivarius I 1794 and Lactobacillus paracasei I 1688 have a direct effect on the immune system, stimulating responses especially on the part of lymphocytes, T regulators and Natural Killer cells which produce a beneficial effect in subjects with a predisposition towards pathologies of the immune system, in particular but not only in allergic and/or immunodeficient subjects.

Thus, according to one of its embodiments, the invention concerns the use of at least one lactobacterium chosen from Lactobacillus salivarius I 1794, Lactobacillus paracasei I 1688 and a mixture thereof, for the preparation of a composition suitable for modulating the immune system.

The Lactobacillus paracasei I 1688 and Lactobacillus salivarius I 1794 were described for the first time in the European patent N^{o} 0861905, granted on Nov. 24, 2004, in the name of the same applicant for the treatment of disorders of the intestinal system.

“Mixture” according to the present invention indicates an association of the two strains of lactobacteria mentioned above, in any relative proportion.

The expression “modulate the immune system” means, according to the present invention, that the composition of the invention is able to stimulate certain responses, of the immune system making it more reactive, for example intervening, via the production of specific cytokines, in development of the cells involved in the immune response.

According to a preferred embodiment, the mixture of the invention comprises the two lactobacteria specified above in a ratio of approximately 10:1, more preferably in the following respective proportions: 92% Lactobacillus paracasei I 1688 and 8% Lactobacillus salivarius I 1794.

The mixture comprising 92% Lactobacillus paracasei I 1688 and 8% Lactobacillus salivarius I 1794 is known per se for the treatment of disorders of the gastrointestinal tract such as dysmicrobism and is commonly and commercially called “PS MIX” (trademark application).

Other mixtures comprising different relative quantities of the two lactobacteria, if necessary combined with other lactobacteria or appropriate active agents, can also be used according to the invention.

The Lactobacillus salivarius I 1794, the Lactobacillus paracasei I 1688 and their mixtures as defined above, hereinafter also defined “active ingredients”, are particularly useful as medicaments in the prevention and treatment of pathologies associated with alterations of the immune system.

It has in fact been demonstrated that said active ingredients are active in the stimulation of different immune cell types, more specifically in pathologies that involve a response of the lymphocytes, especially T-helper and T-cytotoxic lymphocytes with CD25, Natural Killer cells, B lymphocytes, dendritic cells and cytokines including, for example, TNFα, IFNγ, IL-10 and IL-12.

The details of the tests performed and the surprising results obtained with the active ingredients of the invention are given in the experimental section of the present description.

The Lactobacillus salivarius I 1794, the Lactobacillus paracasei I 1688 and their mixtures can therefore be used for example in the treatment and prevention of allergies and resulting pathologies.

The Lactobacillus salivarius I 1794, the Lactobacillus paracasei I 1688 and their mixtures can also be used in the treatment and prevention of immunodeficiencies Of any origin and resulting pathologies.

Thus, according to another of its embodiments, the invention concerns the use of the compositions of the invention, advantageously of the compositions in oral form, for the treatment of bacterial or viral infections, such as infections of the respiratory tract, infections of the gastrointestinal tract, infections of the mucous membranes, infections of the skin and all infections deriving from states of immunodeficiency.

In a condition of immunodeficiency, the defences of the organism against pathogens are reduced with consequent alteration of the Th1/Th2 balance as, for example, in physiological immunodeficiencies (newly-born babies, pregnancy), congenital immunodeficiencies (genetic diseases) and acquired immunodeficiencies (AIDS, autoimmune diseases).

The compositions of the invention can also be useful to support the natural immune defences of the organism, for example in particular states of stress such as psychophysical stress which, if excessively intense or protracted, can lead to a situation of immunodeficiency, clinically manifested by infectious forms of varying intensity.

To perform their action, the active ingredients of the invention, i.e. the Lactobacillus salivarius I 1794, the Lactobacillus paracasei I 1688 and their mixtures are preferably administered systemically, advantageously orally, in the form of compositions, possibly but not necessarily combined with one or more physiologically and/or pharmaceutically acceptable excipients or vehicles.

The term “compositions”, according to the present invention, indicates any composition whether pharmaceutical, dietetic, alimentary or nutraceutic, advantageously oral, which comprises the lactobacteria described above or mixtures thereof.

Said compositions axe prepared according to the known technique, taking account of the particular nature of the active ingredients of the invention consisting of living material, i.e. lactobacteria, which must therefore be treated so that it is able to survive processing, storage and administration.

According to a preferred embodiment, the compositions for use according to the invention are in the form of dosage units for administration once or several times a day, according to the type and severity of the pathology to be treated and the age and weight of the patient. In general said dosage units contain between 10³ and 10¹², advantageously between 10⁵ and 10¹⁰, for example between 10⁸ and 10¹⁰ CFU (colony forming units) of active ingredients per gram of composition.

For an adequate treatment, 1 to 3 dosage units, for example, are generally administered per day.

The active ingredients for use according to the invention are preferably administered orally and are in lyophilised form, included in suitable compositions possibly but not necessarily with excipients and conventional stabilisers, according to the methods well known to a person skilled in the art.

Thus for use according to the invention, the compositions containing the active ingredients are administered for an appropriate period of time, if necessary established by the specialist in charge, which varies in general between 1 week and 1 month or even more. Shorter or longer treatments can be taken into consideration, also in view of the non-toxicity of the active ingredients which permits the use thereof even in excess without dangerous side effects on the health of the subject treated.

The pharmaceutical, dietetic, alimentary and nutraceutic compositions according to the invention include any medicament, food, dietetic or nutraceutic product able to provide a vehicle for the lactobacteria described above in the organism. For purely illustrative purposes, these include, for example, drugs; dietetic products; products deriving from milk, such as yoghurt, cheese, cream; confectionery; fruit juices; etc..

Said compositions can comprise other beneficial substances for the organism such as, for example, vitamins, mineral salts, and/or other compatible active ingredients, for example prebiotic agents such as inulins, phospho-oligosaccharides (FOS), fibres, etc.

The invention also concerns a method for the prevention and treatment of pathologies of the immune system as defined above, which comprises administering to a mammal an effective dose of an active ingredient chosen from Lactobacillus salivarius I 1794, Lactobacillus paracasei I 1688 and a mixture thereof.

The following examples illustrate the invention without limiting it in any way.

### EXAMPLE 1

### Evaluation of direct activity on the cells of the systemic immune system of a mixture of Lactobacillus salivarius I 1794 and Lactobacillus paracasei I 1688

The proliferative response of the lymphocytes of the peripheral blood of some subjects as a response to the following doses of Lactobacillus salivarius I 1794 and Lactobacillus paracasei I 1688 and to two mixtures of the same was evaluated.

Before proceeding with the different evaluations described below, the bacteria underwent gamma irradiation for 24 hours with a source of cesium in order to block all spontaneous proliferation of the same.

The lymphocytes of 15 healthy subjects, isolated by Fycoll-Hypaque density gradient centrifugation (Lymphoprep, Nycomed Pharma AS, Oslo, Norway), re-suspended in an RPMI 1640 medium (Gibco BRL, Life Technologues, Paisley, Scotland) with the addition of 2 mM of L-glutamihe, 50 μg/m of gentamicin (Gibco) and 10% of fetal calf serum (FCS, Euroclone-Celbio, Milan, Italy) (RPMI-FCS) were placed in a culture (1×10⁵/200 μl) in triplicate, in microplates with 96 U-bottom wells (non-stimulated cultures) or (stimulated cultures) with Lactobacillus salivarius (I 1794) (1×10⁵/200 μl), Lactobacillus paracasei (I 1688) (1×10⁵/200 μl), PS MIX (trademark application) (1×10⁵/200 μl), or Candida albicans (2×10⁵/200 μl) as a positive control, at 37° C. in a humidified atmosphere, with 5% of CO₂. Eighteen hours after collection, 1 μCi of [³H]TdR (Amershampharmacia Biotec, Milan, Italy) was added to each well.

The following ratios between bacteria/peripheral blood lymphocytes were used for the proliferation tests:

bacteria:lymphocytes
200:1
100:1
20:1
10:1
5:1
1:1
0.5:1
0.1:1
0.01:1
0.005:1
0.001:1
   compared with the proliferative response of Candida Albicans at two ratios (02:1 and 2:1) in a six-day cell culture, at 37° C. in an atmosphere with 5% of CO₂.

The results were expressed as Stimulation Index (SI: cpm of stimulated cultures/cpm of non-stimulated cultures, where cpm means “counts per minute”) and indicate measurement of the recognition by the lymphocytes of the bacterial antigens and the ability to respond, by proliferating, to the antigen recognition.

### Results

An excellent proliferative response to the bacteria/cell co-culture was observed denoting recognition by the lymphocytes of the antigen determinants of the lactobacteria and lymphocyte activation following said recognition.

### EXAMPLE 2

### Phenotype of the Lymphocytic Subpopulations

The phenotype of the lymphocytic subpopulations was evaluated at time 0, before culture of the bacteria, and after 6 days' culture to evaluate which phenotype was induced.

Monoclonal antibodies anti-CD3, anti-CD4, anti-CD8, anti-CD25, anti-HLA were used to evaluate the T lymphocytic populations and subpopulations, anti-CD16 and anti-CD56 to evaluate the Natural Killers, anti-CD20, anti-CD38 and anti-CD79 for the lymphocytic B populations and the plasmacells. The reading was performed with a tricolour method.

### Results

A substantial increase in the Natural Killers was observed, which, as is known, provide a better response to the cells infected by viruses and the neoplastically transformed cells.

An increase in the T-lymphocyte helpers and cytotoxic T-lymphocytes with CD25 membrane, which represent the subpopulations most directly affected by a modulation of the immune response, was also observed.

### EXAMPLE 3

The production of some cytokines was ascertained in the buffy coat of the cell cultures via an ELISA method developed for each cytokine (TNF-alfa, IFN-gamma, IL-10, IL-12, IL-4).

### Results

The production of IL-4 in the supernatant in response to activation of the lymphocytes by the lactobacteria was below the levels measurable with the ELISA test, indicating non-activation of the T-helpers 2 (Th2) responsible for induction of the allergenic phenomena.

High TNF-alfa and IFN-gamma values were found, on the other hand, indicating activation of the T-helpers 1 (Th1).

Considerable quantities of IL-10 and IL-12 were also found; as is known, the first induces differentiation of the B lymphocytes, inhibits activation of the macrophages and protects against the risk of inflammatory bowel disease (IBD), and the second activates the Natural Killers inducing the production of IFN-gamma, a crucial agent in the first phases of an infection.

### EXAMPLE 4

An oral composition is prepared, in powder form, comprising the following components for each dosage unit:

| | |
|---|---|
| PS MIX (trademark application) (lyophilised) | 1 × 10⁸-1 × 10¹⁰ |
| Sucrose; Malt dextrins; Aroma; Silica; Vitamin B2; | |
| Vitamin B1. | |

1-15. (canceled)

16. A method for the prevention and treatment of pathologies of the immune system comprising administering to a mammal an effective dose of an active ingredient chosen from Lactobacillus salivarius I 1794, Lactobacillus paracasei I 1688 and a mixture of Lactobacillus salivarius I 1794 and Lactobacillus paracasei I 1688.

17. The method of claim 16 characterised in that said mixture of Lactobacillus salivarius I 1794 and Lactobacillus paracasei I 1688 is used, in any proportion.

18. The method of claim 17, characterised in that said mixture comprises 8% of Lactobacillus salivarius I 1794 and 92% of Lactobacillus paracasei I 1688.

19. The method according to claim 16 wherein said prevention and treatment of pathologies of the immune system is associated with an alterations of the function of T-helper CD25 lymphocytes, T-cytotoxic CD25 lymphocytes, Natural Killer cells, B lymphocytes, dendritic cells and cytokines.

20. The method of claim 19 wherein the cytokine is selected from the group consisting of TNFα, INFγ, IL-4, IL-10 and IL-12.

21. The method of claim 16 for the prevention and treatment of allergies.

22. The method of claim 16 for the prevention and treatment of immunodeficiencies and pathologies deriving therefrom.

23. The method of claim 16 wherein said Lactobacillus salivarius I 1794, Lactobacillus paracasei I 1688 and a mixture of Lactobacillus salivarius I 1794 and Lactobacillus paracasei I 1688 are administered in the form of oral compositions if necessary with one or more physiologically and/or pharmaceutically acceptable excipients and/or vehicles.

24. The method of claim 16 wherein said Lactobacillus salivarius I 1794, Lactobacillus paracasei I 1688 and a mixture of Lactobacillus salivarius I 1794 and Lactobacillus paracasei I 1688 are administered in the amount of at least 1×10⁸-1×10¹⁰ CFU per day.

25. The method of claim 16 wherein said Lactobacillus salivarius I 1794, Lactobacillus paracasei I 1688 and a mixture of Lactobacillus salivarius I 1794 and Lactobacillus paracasei I 1688 are administered after reconstitution of a lyophilised form.

26. The method of claim 16 wherein said Lactobacillus salivarius I 1794, Lactobacillus paracasei I 1688 and a mixture of Lactobacillus salivarius I 1794 and Lactobacillus paracasei I 1688 are administered in the form of dosage units.

27. The method of claim 16 for the treatment of respiratory infections, infections of the gastrointestinal tract, infections of the mucous membranes, infections of the skin, or infections deriving from a state of immunodeficiency.
